(19) United States
(12) Patent Application Publication
(10) Pub. No.: US 2010/0028407 A1
(43) Pub. Date: Feb. 4, 2010

(54) LAYERED BIO-ADHESIVE COMPOSITIONS AND USES THEREOF

(75) Inventors: Lucian V. Del Priore, Basking Ridge, NJ (US); Henry J. Kaplan, Louisville, KY (US); Tongalp Tezel, Louisville, KY (US)

Correspondence Address: WilmerHale/Columbia University, 399 PARK AVENUE, NEW YORK, NY, 10022, US

(73) Assignee: UNIVERSITY OF LOUISVILLE RESEARCH FOUNDATION, INC., Louisville, KY (US)

(21) Appl. No.: 12/298,698

(22) PCT Filed: Apr. 24, 2007
(86) PCT No.: PCT/US07/09861

§ 371 Date: Aug. 10, 2009

Related U.S. Application Data
(60) Provisional application No. 60/795,437, filed on Apr. 27, 2006.

(51) Int. Cl. A61K 35/44; C09J 7/02; A61K 9/70; A61F 13/00; A61P 27/02; A61K 47/42; A61K 47/36; A61K 47/34; A61K 47/26; A61P 17/02

(52) U.S. Cl. 424/443; 428/337; 424/93.7; 514/772.3; 514/772.7; 514/774; 514/773; 514/777; 523/118

(57) ABSTRACT

The invention generally provides compositions and methods for promoting and enhancing wound closure and healing. Specifically, the invention provides a biologic composition which comprises a support layer which serves as transport scaffold, for example made of gelatin, which is coated or impregnated with a bio-adhesive molecule such as rose bengal or glyceraldehyde. The composition can also comprise an artificial or biological matrix, optionally processed (i.e. cleaned and coated with extracellular matrix proteins) to enhance cell attachment and survival. The composition can further comprise a monolayer of epithelial, endothelial cells or mesenchymal cells. The invention provides methods for using the compositions for treating wounds due to disease, trauma or surgery. Specific methods for treating ocular wounds are provided.

This application claims priority to U.S. Ser. No. 60/795437 filed on Apr. 27, 2006 the content of which are hereby incorporated in its entirety.

### BACKGROUND OF THE INVENTION

Joining of separated tissue as a result of surgery or injury to the tissue, or during tissue transplantation is traditionally done by suturing or stapling of tissue. There are problems associated with these techniques, for example sutures or staples permit leakage of fluid and access of microorganism. Therefore, there is need for sutureless repair of wounds such as by use of bio-compatible adhesive molecules or glues which adhere to tissues and form a bond between separated tissues. The invention provides bio-adhesive compositions which are applicable in wound healing and repair, tissue grafting and transplantation.

### SUMMARY OF THE INVENTION

In one aspect, the present invention is directed to a composition comprising: (a) at least one support layer impregnated or coated with a bio-adhesive agent. In certain embodiments, the support layer is coated with a bio-adhesive agent on one side. In other embodiments of the inventive composition, the support layer is coated with a bio-adhesive agent on both sides. The support layer can serve as a transport scaffold for the bio-adhesive agent. The support layer can also comprise sucrose, or any other component, which imparts desirable characteristic, example rigidity, of the support layer, which can be made of gelatin.

In another aspect, the present invention is directed to a composition comprising: (a) at least one support layer which can be impregnated or coated with a bio-adhesive agent, and further comprising (b) an additional support layer, which comprises a matrix which facilitates wound healing. In certain embodiments, the additional support layer can be coated or impregnated with a bio-adhesive agent. In another aspect, the present invention is directed to composition comprising: (a) at least one support layer impregnated or coated with a bio-adhesive agent, and further comprising (b) a matrix which facilitates wound healing.

In certain embodiments of the invention composition, the thickness of the support layer can be from about 1 micron to about 1000 microns, to about 900 microns, to about 800 microns, to about 700 microns, to about 600 microns, to about 500 microns, to about 400 microns, to about 300 microns, to about 200 microns, to about 100 microns.

In certain embodiments, the support layer of the inventive composition comprises material selected from, but not limited to, the group consisting of: gelatin, collagen, poly(ethylene glycol)-block-poly(epsilon-caprolactone)-block-poly(DL-lactide), PEG-PCL-P(DL) lactic acid, RGD-containing peptides (Arg-Gly-Asp) on a polyvinyl alcohol (PVA) surface or glycol-polymer matrix, heparin, alginate cross linked gels, agarose hydro-gels or any combination thereof. The components of the support layer, are provided in concentrations such that the inventive composition is suitable for use in wound closure and tissue bonding. In certain embodiment, wherein the support layer comprises gelatin, the support layer can further comprise sucrose, or any other agent which can impart advantageous characteristics of the support layer such as rigidity and firmness at room temperature, and ability to melt and release the bio-adhesive agent when placed in contact with the wound.

In certain embodiments, the matrix of the inventive composition comprises molecules selected from, but not limited to, the group consisting of: laminin, collagen, fibronectin, vitronectin or any combination thereof. In other embodiments, the matrix of the inventive composition comprises amniotic membrane, human sclera, human cornea, or other basement membranes. In other embodiments, the matrix of the inventive composition consists of 85% collagen and 15% laminin.

In certain embodiments, the matrix of the inventive composition has a thickness which is from about 1 micron to about 500 microns, from about 1 micron to about 1000 microns.

In certain embodiments, the inventive composition further comprises a monolayer of epithelial cells, wherein in a non-limiting example the epithelial cells can be retinal pigment epithelial (RPE) cells. In other embodiments, the inventive composition further comprises a monolayer of endothelial cells. In other embodiments, the inventive composition further comprises a monolayer of mesenchymal cells.

In another aspect, the invention is directed to a composition comprising a bio-adhesive agent, wherein the composition is useful for wound closure, tissue bonding and tissue grafting. The bio-adhesive agent is provided at a concentration which is suitable for use in wound healing and tissue bonding applications. In certain embodiments, the bio-adhesive agent is selected from the group consisting of photo-activated molecules or chemically-active molecules. In certain embodiments, the bio-adhesive agent is a photo-activated molecule, which is selected from, but not limited to, the group consisting of: flavins, xanthenes, thiazines, porphyrins, chlorophyllin and photo-activated derivatives thereof In other embodiments, the flavin photo-adhesive agent is selected from the group consisting of: riboflavin, riboflavin-5-phosphate, flavin mononucleotide, flavin adenine dinucleotide, flavin guanine nucleotide, flavin cytosine nucleotide, flavin thymine nucleotide. In other embodiments, the xanthene photo-adhesive agent is selected from the group consisting of: rose bengal, erythrosine. In other embodiments, the thiazine photo-adhesive agent is selected from the group consisting of: methylene blue. In other embodiments, the porphyrin photo-adhesive agent is selected from the group consisting of: protoporphyrin I through protoporphyrin IX, coproporphyrins, uroporphyrins, mesoporphyrins, hematoporphyrins and sapphyrins. Non-limiting example of chlorophylis is bacteriochlorophyll A.

In certain embodiments, the bio-adhesive agent is a chemically-active adhesive molecule, which can be selected from, but is not limited to, the group consisting of: D-glyceraldehyde, L-glyceraldehyde, glyceraldehydes-3-phosphate, glutareldehyde, glycoaldehyde, oxoaldehydes such as glyoxal and methylglyoxal, dihydroxyacetone, threose, D-xylose, D-ribose, D-fructose, D-glucose, poly(acrylates), chitosan, cellulose derivatives, hyaluronic acid derivatives, pectin and traganth, starch, poly(ethylene glycol), sulfated polysaccharides, carrageenan, Na-alginate, gelatin, theorems.

In another aspect, the present invention is directed to methods for promoting tissue bond formation between separate tissues, the method comprising:
a) providing a composition which comprises a support layer impregnated or coated with a bio-adhesive agent which can lead to tissue bonding,
b) applying the composition to tissues to be bonded,
c) and wherein the composition comprising a bio-adhesive agent which is photo-active is optionally treated by applying electromagnetic energy to the composition to promote tissue bond formation.

In certain embodiments of the methods, the tissues to be bonded are in the eye. In other embodiment, the tissues to be bonded are in an ocular wound due to trauma, surgery, transplantation, disorder or disease. In non-limiting examples, the disorder is selected from the group consisting of: age-related macular degeneration, disorder affecting the RPE-Bruch's membrane complex, presumed ocular histoplamosis syndrome, myopic maculopathy, ingrowth of revascularization from a disorder affecting Bruch's membrane. In non-limiting examples, the ocular wound is selected from the group consisting of: corneal wound, iris wound, scleral wound, an anterior wound following glaucoma surgery, ocular adnexa wound, orbital wound, trabeculectomy, wound produced by tube implants, virectomy incision wound, subretinal fluid drainage wound, orbital surgery wound, lid surgery wound, scleral laceration or perforation, corneal laceration or perforation, wounds due to glaucoma implants and surgery, wounds due to the structure of the sinuses and lid margins, wound due to damage or defects in the integrity of the retinal pigment epithelial-Bruch's membrane complex. In non-limiting examples, the corneal wound is cataract surgery wound, penetrating or lameral keratoplasty surgery wound, scalpel or laser-induced refractive surgery wound. In non-limiting examples, the retinal wound is selected from the group consisting of: retinal hole in the periphery, retinal hole in the macula.

In certain embodiments of the inventive method, the tissues to be bonded are in the skin. In other embodiments, the tissues to be bonded are in blood vessels. In other embodiments, the tissues to be bonded are in deep tissue layers.

In another aspect, the present invention provides methods for transplantation of retinal pigment epithelial cells to a Bruch's membrane of a host's eye, the method comprising:
a) harvesting or obtaining retinal pigment epithelial cells from a donor tissue;
b) applying a composition as described in any of the embodiments of the invention to host's Bruch's membrane,
c) positioning the retinal pigment epithelial cells of step (a) onto the composition of step (b),
d) bonding the composition of step (b) to host's Bruch's membrane, wherein a composition comprising a bio-adhesive agent which is photo-active is optionally treated by applying electromagnetic energy to the composition to promote tissue bond formation

In certain embodiments of the method for transplantation, the retinal pigment epithelial cells which are harvested from the donor are cultured on a culture substrate to form a monolayer of cells.

In another aspect, the present invention provides a kit comprising any one of inventive compositions. In certain embodiments, the compositions are dispensed into light-impenetrable container, and also comprise a pharmaceutically acceptable carrier. In certain embodiments, wherein the bio-adhesive molecule is a chemically active molecule, the chemically active molecule is provided separately from the remaining components of the composition. In certain embodiments, the kit comprises a composition wherein the area of the support layer is predetermined.

In another aspect, the present invention provides a method for making a bio-adhesive composition, the method comprising:
a) providing a gelatin block comprising about 50% gelatin, and optionally comprising sucrose;
b) sectioning a gelatin sheet from the gelatin block; wherein the gelatin sheet is from about 1 micron to about 1000 microns.
c) impregnating or coating the gelatin sheet with a bio-adhesive agent, thereby creating a bio-adhesive composition.

In certain embodiments, the gelatin has rigidity of 175 Blooms, 225 Blooms or 300 Blooms. In other embodiments, the gelatin block comprises from about 10% to about 50% gelatin, to about 60%, to about 70%, to about 85% gelatin, to about 95% gelatin. In certain embodiment, the gelatin block comprises about 10% gelatin, about 15% gelatin, about 20% gelatin, about 25% gelatin, about 30% gelatin, about 35% gelatin, about 40% gelatin, about 45% gelatin, about 55% gelatin, about 60% gelatin, about 65% gelatin, about 70% gelatin, about 75% gelatin, about 80% gelatin, about 85% gelatin, about 90% gelatin In certain embodiments, the bio-adhesive agents is selected from the group consisting of photo-activated or chemically-active molecules. In various embodiments, the amount of sucrose can also be varied to achieve specific. rigidity requirements of the gelatin sheet, and the gelatin block from which the gelatin sheet was sectioned.

In certain embodiments of the method of making the gelatin block, the gelatin is sterilized prior to dissolving into solution. In one embodiment, gelatin is sterilized by gamma irradiation. Gelatin can be exposed a gamma source receiving irradiation in a range from about 100 krad to about 4 Mrad, depending on the duration of exposure to the gamma ray source. In non-limiting examples, gelatin is irradiated with 1.2 Mrad, or 2.7 Mrad.

Additional aspects of the invention will be apparent in view of the description which follows.

### BRIEF DESCRIPTION OF THE FIGURES

FIG. 1 depicts one embodiment of the composition of the invention. Part 1, marked as Layer 1, is a support layer which also serves as a transport scaffold material which is coated or impregnated with a photo-activated or chemically active bio-adhesive molecule. This embodiment further comprises Part 2, marked as Layer 2, which is an artificial or biological matrix, which can be optionally processed (i.e. cleaned and/or coated with extracellular matrix proteins) to enhance cell attachment and survival. This embodiment further comprises Part 3, marked as Layer 3, which is a monolayer of epithelial, endothelial cells or mesenchymal cells.

FIG. 2 depicts representative results that demonstrate the attachment strength of repaired wounds, wherein the damaged tissue in the wound was bonded by compositions of the invention.

FIG.3 depicts representative results showing that photo-melding of tissue, through photo-activation of photo-adhesive molecules, does not affect cell viability.

FIG.4 depicts representative results showing that photo-melding of tissue, followed by additional exposure to ambient light, does not affect cell viability.

### DETAILED DESCRIPTION OF THE INVENTION

As used herein a “support layer” is a biological material which provides solid support for other components, such as bio-adhesive molecules, optional matrix and optional monolayer of cells, of the inventive composition. In certain embodiments, the support layer is coated or impregnated with bio-adhesive molecule, thus serving as a transport scaffold for delivery of the bio-adhesive molecule.

“Bio-adhesive” or “tissue bonding” molecules, refer to molecules which are biologically compatible with tissues and which can produce a bond between abutting tissues which are exposed to the bio-adhesive molecule. The terms bio-adhesive molecules, compounds or agents, and tissue bonding molecules, compounds or agents are used interchangeably. The term bond refers to a structural and functional connection between two tissues which were physically separated, for example, by a surgical incision, tissue trauma, or during tissue grafting or transplantation.

The term “photo-activated bio-adhesive” refers to molecules which can undergo photo-activation. Photo-activation is a process by which energy in the form of electromagnetic radiation is absorbed by a compound which becomes “excited” and then converts the energy to another form of energy, preferably chemical energy. The chemical energy can be in the form of reactive oxygen species like singlet oxygen, superoxide anion, hydroxyl radical, the excited state of the photo-activated molecule, photo-activated free radical or substrate free radical species. The electromagnetic radiation will include “optical energy”, i.e., can have a wavelength in the visible range or portion of the electromagnetic spectrum, and can also include the ultra violet and infra red regions of the spectrum. Photo-activation processes of interest in the invention are those which involve reduced, negligible, or no conversion or transfer of the absorbed energy into heat energy. Photo-activation of photo-adhesive molecules leads to photo-melding of tissues exposed to the photo-adhesive molecules.

As used herein the term “matrix” refers a component of the inventive composition. The term matrix refers to an artificial or biological material which functions to enhance tissue bonding, cell adhesion and cell repopulation during wound healing and tissue transplantation.

“Monolayer” of cells refers to a monolayer of epithelial, endothelial or mesenchymal cells, which can be a component of the inventive composition.

Traditional methods of wound closure, such as suturing, are not well suited to all tissues and are frequently associated with complications including foreign body immunological response and infection. The ideal wound closure method would be rapid, non-invasive and yield a strong water-tight seal between the bonded tissues, and would not affect the structural and/or functional integrity of the bonded tissue. Retaining tissue structural integrity during tissue bonding is particularly important in eye tissues, for example the cornea, where any significant tissue deformation will induce astigmatism and increase the chance of endophthalmitis. Retaining tissue structural integrity during tissue bonding is also important in healing of wounds after cataract surgery and in the closure of wounds following vitreous surgery (i.e., vitrectomy). Furthermore, retaining the integrity of Bruch's membrane is important since RPE may sense the elasticity and rigidity of its matrix.

In view of the foregoing, the invention provides compositions and methods to facilitate and improve tissue bonding in wound treatment and closure, and tissue transplantation. In certain embodiments, the compositions of the invention are suitable for the treatment of oculars wound associated with or caused by: ocular disorders, trauma and surgery, ocular wounds in the anterior segment of the eye, such as cataract wounds, scleral and corneal lacerations and perforations, penetrating keratoplasty surgery, glaucoma implants, trauma and surgery to the eye. In other aspects, the compositions of the invention are used for the treatment of wounds to the skin, face and ocular adnexa, and orbital wounds; wounds related to the structures of the face, such as the nose and nasal sinuses and lids margins, restoring the integrity of the RPE-Bruch's membrane complex with patch grafts. In other aspects, the inventive compositions can be used to treat wounds elsewhere within the human body, such as closure of skin wounds, vessels, and deep-tissue-layer wounds.

In one aspect, the invention provides compositions and methods for tissue bonding. In another aspect, the invention provides compositions and methods for suturelss wound closure and healing. The invention provides compositions and methods which are useful in tissue grafting and transplantation. The invention further provides compositions and methods which enhance wound closure and healing. In certain embodiments, the compositions of the invention are specifically directed to treating wounds and tissue damage in the eye. In certain embodiments, the tissue damage in the eye can be due to an eye disorder, for example but not limited to macular degeneration. In certain embodiments, the compositions and methods of the invention are useful for treating wounds in the anterior and posterior portion of the eye related to damage incidental to transplantation, surgical incisions, and repairs. In certain embodiments, the compositions and methods of the invention are useful for treating wounds in the anterior and posterior portion of the eye due to damage incidental to trauma and injury.

In one aspect, the invention provides a biocompatible composition, which comprises at least one support layer. The support layer can serve as a transport scaffold, which carries a bio-adhesive molecule. In certain embodiments, the biocompatible composition consists essentially of one support layer, wherein the support layer carries a bio-adhesive molecule. The bioadhesive molecule can be impregnated in the support layer.

One function of the support layer is to serve as a scaffold and provide structural integrity of the biocompatible composition. In certain embodiments, one support layer can confer structural support. In other embodiments, structural support can be conferred by multiple support layers, which can be included in more than component of the inventive composition.

Another function of the support layer is to serve as a carrier and transport scaffold for the bio-adhesive molecule. In another aspect, the scaffold provides solid support for the bio-adhesive molecule. In one embodiment, the support layer is impregnated with a bio-adhesive molecule. In another embodiment, the support layer is coated with a bio-adhesive molecule. In another embodiment, the support layer is soaked with a bio-adhesive molecule. The bio-adhesive coating can be coated on either side of the support layer, or on both sides of the support layer. The bio-adhesive coating can cover the entire surface of the support layer. Alternatively, the bio-adhesive coating can be applied in a discontinuous manner or pattern on the surface of the support layer. Regardless of the manner of application of the bio-adhesive molecule to the support layer, the bio-adhesive molecule is provided at a concentration that is sufficient to produce tissue bonding between two abutting tissues exposed to the inventive composition.

The scaffold is a major advantage of the inventive composition, because this scaffold allows delivery of the bio-adhesive molecules of interest as a thin film rather than as a solution or a viscous composition. Thus the bio-adhesive molecule is confined to the treatment region, with little spread beyond the treatment area, and thereby providing effective concentration of agents where needed. For example, after cataract surgery the thin multilayer film is placed on or in the wound, wherein tissue cross-linking results in wound closure. Use of the scaffold coated with a bio-adhesive molecule would prevent the spread of the bio-adhesive reagents into the anterior chamber of the eye or loss of the therapeutic agents into the surgical field.

In certain embodiments, the bio-adhesive agent is a photo-activated bio-adhesive molecule. Non-limiting examples of photo-activated compounds are flavins, xanthenes, thiazines, porphyrins, expanded porphyrins, chlorophylis and chlorophyllin, and photo-activated derivatives thereof. Non-limiting examples of flavins are riboflavin, riboflavin-5-phosphate, or flavine mononucleotide, flavin adenine dinucleotide as well as flavin guanine nucleotide, flavin cytosine nucleotide and flavin thymine nucleotide compounds. Non-limiting examples of xanthenes are rose bengal and erythrosine. Non-limiting example of thiazines is methylene blue. Non-limiting examples of porphyrins and expanded porphyrins are: protoporphyrin I through protoporphyrin IX, coproporphyrins, uroporphyrins, mesoporphyrins, hematoporphyrins and sapphyrins. Non-limiting example of chlorophylis is bacteriochlorophyll A. These compounds can be utilized in the mono-, di- and tri-phosphorylated species.

Photo-activated compounds exhibit their adhesive properties upon activation by exposure to an appropriate energy or light source. The specific conditions for activation of the above identified photo-activated compounds are well known in the art. Usually, photo-activation will require a wavelength from about 10 nm to about 1064 nm and will be within the visual, infrared or ultra violet spectra. The radiation can be supplied in the form of a single or dual monochromatic laser beam(s), filtered light or other form of electromagnetic radiation source. The choice of energy source depends on the photo-activated molecule employed in the composition. For example, an argon laser is particularly suitable for use with flavins such as riboflavin-5-phosphate, i.e., flavins are optimally excited at wavelengths corresponding to the wavelength of the radiation emitted by the argon laser. A diode laser is particularly suitable for use with chlorophylis such as bacteriochlorophyll A, and an excimer laser is suitable for refractive surgeries. Broad band white light, which can be filtered to a narrow range of wavelengths may also be used for photoactivation. Suitable combinations of energy sources and photo-activated molecule are known to the skilled artisan. Photo activation preferably occurs with no more than a 1-2° C. rise in temperature, preferably no more than 1° C. rise and more preferably no more than 0.5° C.

In other embodiments, the bio-adhesive agent is a chemically-active adhesive molecule. Non-limiting examples of chemically active bio-adhesive molecule are: D-glyceraldehyde, L-glyceraldehyde, glyceraldehydes-3-phosphate, glutaraldehyde, glycoaldehyde, oxoaldehydes such as glyoxal and methylglyoxal, dihydroxyacetone, threose, D-xylose, D-ribose, D-fructose, D-glucose, and chemically active derivatives thereof. Chemically active bio-adhesive molecules lead to tissue bonding which is generally a spontaneous process. Providing chemically active bio-adhesive molecules by coating or impregnating gelatin sheets with different physicochemical properties is advantageous because it allows to modify the concentration and release rate of the chemically active bio-adhesive, and/or to control the rate of the chemical reaction that results in tissue bonding. Thus the support layer can also control the rate of release of the tissue bonding agent. Controlled rate of release can control the rate of tissue bonding. Controlling the rate of the chemical reaction that leads to tissue bonding may be useful in various different wound closure settings. In certain embodiments of this invention, a non-limiting example is the closure of vitrectomy wounds, the surgeon may prefer rapid wound closure to avoid leak of fluid or gas or silicone oil from the vitreous cavity into the subTenon's or subconjunctival space. In this case, a relatively thick, about 200 μm, sheet of 10% gelatin soaked with, for example but not limited to, glyceraldehyde can melt immediately upon contact with the wound, releasing the glyceraldehyde and resulting in wound closure. In another embodiment, a small 20-30 gauge plug consisting of the composition described herein can be adapted to be inserted into a varectomy wound, the composition can melt after insertion into the wound and thus result in wound closure. In another embodiment, the closure of corneal incisions during cataract surgery can require placing a relatively thin, about 50 μn, sheet containing 50% gelatin, impregnated or coated with a chemically active bio-adhesive, between the wound lips. In this case, the 50% gelatin sheet will plug the wound initially. Because of the higher gelatin concentration, it will take longer for this gelatin sheet to melt away and to release the chemically active bio-adhesive which will eventually crosslink the collagen at the wound lips. In another embodiment, the closure of a wound during cataract surgery can require placing across the wound lips a thin sheet, about 50 micron sheet comprising less than 50% gelatin, which sheet is impregnated or coated with a chemically active bioadhesive molecule. Rapid melting of the gelatin with release of the bioadhesive molecule will cross-link the collagen across the wound lips.

In certain embodiments, wherein the scaffold comprises gelatin, the gelatin concentration may vary from about 10% to about 20%, 30%, 40%, 50%, 60%, 70%, 80%, 90%. That composition can be supplemented with an additional component, for example but not limited to 300 mM of sucrose, wherein the additional component increases the rigidity and thermal relaxation time window for wound closure. In certain embodiments, the gelatin scaffold may be impregnated with one or more of the following molecules: Rose Bengal, glyceraldehydes, riboflavin. Rose Bengal can be impregnated at 0.5 mM concentration, wherein the range of Rose Bengal concentration is from about 0.5 mM to about 6.0 mM, to about 5.5 mM, to about 5 mM, to about 4.5 mM, to about 4.0 mM, to about 3.5 mM, to about 3.0 mM, to about 2.5 mM, to about 2.0 mM, to about 1.5 mM, to about 1.0 mM. Glyceraldehyde can be impregnated at 0.2 mM concentration, wherein the range of glycerlaldehyde concentration can vary from about 0.05 mM to about 2.0 mM, to about 1.5 mM, to about 1.0 mM, to about 0.5 mM, to about 0.1 mM. The concentration of riboflavin is 0.1%, wherein the range of riboflavin concentration is from about 0.01% to about 1%, to about 0.9%, to about 0.8%, to about 0.7%, to about 0.6%, to about 0.5%, to about 0.4%, to about 0.3%, to about 0.2%, to about 0.1%, to about 0.05%, to about 0.02%.

In certain embodiments, the invention provides compositions, which comprise a combination of two or more photo-activated bio-adhesive molecules. In other embodiments, the invention provides composition, which comprise a combination of two or more chemically active bio-adhesive molecules. In other embodiments, the invention provides compositions, which comprise a combination of at least one photo-active bio-adhesive and at least one chemically active photo-adhesive molecule. A composition, which comprises a combination of photo and chemically active bio-adhesive can start forming a tissue bond due to the photo-activation of the photo-adhesive molecules, and further maintain the tissue bond by the action of the chemically active adhesive. In other non-limiting examples, a combination which comprises a bio-adhesive and a chemically active agent may be useful in a treatment course where the eye is covered with a patch immediately after the surgery and where the patch is removed the next day during the first postoperative visit. In such treatment, a combination of a chemically active and photo-activated bio-adhesive molecule may be used to seal the wound. The chemically activated chemical (for example, glyceraldehyde) may initiate closure of the wound while the eye is patched, and the photo-activated chemical (for example, Rose Bengal) may further strengthen wound closure due to the chemical activation by light when the wound is exposed to light. Such combination of chemically active and photo-active bio-adhesive can allow for an additive effect to the process of wound closure due to the light-activated tissue bonding.

In certain embodiment, the support layer is impregnated or coated with photo-activated bio-adhesive molecule well in advance of application of the composition. In other embodiments, wherein the support layer carries a chemically active bio-adhesive molecule, the support layer is coated or impregnated with the chemically active bio-adhesive immediately before application of the composition to the tissues to be bonded. Coating or impregnating of the support layer with a chemically active bio-adhesive well in advance before the application of the composition to tissue may not be desirable. Prolonged contact of the support layer to the chemically active bio-adhesive may result in an undesirable cross-linking of molecules in the support layer. Therefore in certain embodiments of the invention, the compositions of invention will be provided in a kit, wherein the bio-adhesive molecule, specifically a chemically active molecule is provided separately from the rest of components of the inventive composition. The support layer of such composition can be coated or impregnated with the chemically active molecule, shortly before the application of the bio-adhesive composition to the tissue to be bonded.

In accordance with the invention, the support layer is made from a material that will not impede normal tissue function. The support layer can be solid, a solid or viscous gel, or a sol. Suitable materials which can be used in the support layer of the invention include but are not limited to gelatin, collagen, artificial matrices such as synthetic polypeptides including but not restricted to poly(ethylene glycol)-block-poly(epsilon-caprolactone)-block-poly(DL-lactide), PEG-PCL-P(DL) lactic acid, RGD-containing peptides (Arg-Gly-Asp) on a polyvinyl alcohol (PVA) surface or glycol-polymer matrix, heparin, alginate cross linked gels, agarose hydro-gels or any combination thereof. Other materials, which meet the functional requirements of the support layer, are also contemplated by the invention.

In certain embodiments, gelatin is used as the support layer of the inventive composition. Gelatin can be tested and graded according to its strength, by measuring the rigidity of a gelatin film. The Grade is based on the “Bloom” test and the higher the Bloom number, the higher the Grade and the higher the rigidity of the gelatin film. Gelatins of 125 Bloom, 175 Bloom, 225 Bloom, 250 Bloom, 300 Bloom are contemplated for use in the invention.

The specific composition of the inventive scaffold provide unique characteristics of the scaffold that are particularly useful in the invention. In certain embodiments, it is desirable that the composition provides rigidity of the scaffold so that the scaffold can be cut as thin as 50 μm, or about 40 μm, about 30 μm, about 20 μm, or about 10 μm. Use of 300 bloom unit gelatin can provide a scaffold with such rigidity. Gelatin has to be sterilized because most gelatin products are contaminated mainly by Bacillus species. Contamination by Bacillus species can be difficult to remove. A method of the invention demonstrated than removal of the contamination can be achieved by treatment of gelatin with gamma irradiation. In certain embodiments, irradiation is performed overnight with 1 Grads of gamma radiation. In other embodiments, irradiation can be performed for different duration of time, thus exposing gelatin to different amount of gamma irradiation. Exposure to gamma irradiation can be anywhere from about 100 rads to about 4 Mrads. Exposure to gamma irradiation also breaks collagen and allows to prepare 50% concentration, which otherwise can be difficult to achieve due to low solubility of collagen. Gelatin solution can include other component(s) which increase the rigidity of the gelatin sheets. Non-limiting example of such component is sucrose, for example at 300 mM concentration, which increases the rigidity of gelatin. In various embodiments, sucrose can be added at concentration from about 100 mM to about 1M. Formulations comprising gelatin and sucrose as described herein allow the gelatin sheets to stay as a solid gel which can be manipulated with a forceps, i.e. placing between the wound lips, but to melt within minutes of placing at the wound site thus releasing the impregnated chemical as well as the collagen fragments which helps bridge the wound lips.

Skilled artisans appreciate that one important and formula-specific characteristic of the inventive composition is its ability to remain solid at room temperature and melt within minutes upon contact with body temperature. This makes the inventive composition a highly desirable scaffold because it can be handled easily during the surgery and encase the impregnated chemical until the scaffold melts down at the target site.

In another aspect, the invention provides a biocompatible composition, which comprises a first support layer and a second part, a matrix, which also facilitates tissue bonding. In certain embodiments, the matrix is positioned on top of the first support layer. In other embodiments, the matrix is positioned on top of an additional support layer. In other embodiments, the matrix is positioned within the additional support layer. In certain embodiments, the matrix can contain a bio-adhesive molecule, wherein the bio-adhesive molecule is photo-activated or chemically active. The additional support layer may contain a bio-adhesive molecule. A support layer is provided by a number of different embodiments described herein.

In certain embodiments, the matrix is an artificial matrix. In other embodiments, the matrix is synthetic. In other embodiments, the matrix is biological. An artificial matrix is created from components such as collagen, laminin, vitronectin, and fibronectin, or mixtures thereof. A biological matrix is a sheet of tissue that is harvested from an organism; examples of biological matrices include but are not limited to amniotic membrane; human sclera from eye bank eyes; human cornea; other basement membranes. In some applications (for example, cataract surgery wound healing) it could be beneficial to use an artificial matrix, whereas in others (macular reconstruction) it may be more beneficial to use human basement membrane such as Bruch's membrane from eye bank eyes. In certain embodiments, the biologic and artificial matrices can be placed on a support layer as described herein.

The matrix of the invention comprises material that will not impede normal tissue function. The matrix of the invention can be artificial or biological matrix. In certain embodiments, the matrix can be optionally cleaned, treated or coated such that the matrix enhances cell attachment and cell survival. In non-limiting examples, the matrix can be cleaned by treatment with Triton X-100 or other acceptable solution. In non-limiting examples, the matrix can be treated and cleaned with a laser including but not limited to visible wavelength and UV lasers, such as an excimer laser, infrared lasers such as a diode laser. In non-limiting examples, the matrix can be coated with laminin, fibronectin, or vitronectin, or therapeutically effective mixtures thereof.

In certain embodiments, the matrix composition comprises collagen and laminin, and other optional compounds, the amount and inclusion of which will depend upon the specific application of the composition. In certain embodiments, the matrix comprises Type IV collagen. In other embodiments, the matrix of the inventive compositions can comprise collagen, laminin (330 μg/ml, range typically but no limited to 10-1000 μg/ml, to 10-900 μg/ml, to 10-800 μg/ml, to 10-700 μg/ml, to 10-600 μg/ml, to 10-500 μg/ml, to 10-400 μg/ml, to 10-300 μg/ml, to 10-200 μg/ml, to 10-100 μg/ml), fibronectin (250 μg/ml, range typically but no limited to 10-1000 μg/ml, to 10-900 μg/ml, to 10-800 μg/ml, to 10-700 μg/ml, to 10-600 μg/ml, to 10-500 μg/ml, to 10-400 μg/ml, to 10-300 μg/ml, to 10-200 μg/ml, to 10-100 μg/ml), and vitronectin (33 μg/ml, range typically but no limited to 5-500 μg/ml, to 5-450 μg/ml, to 5-400 μg/ml, to 5-350 μg/ml, to 5-300 μg/ml, to 5-250 μg/ml, to 5-200 μg/ml, to 5-150 μg/ml, to 5-100 μg/ml, to 5-50 μg/ml). In other embodiments, the matrix can include laminin (330 μg/ml), (fibronectin (250 μg/ml), and vitronectin (33 μg/ml). In other embodiments, the matrix can include collagen, fibronectin (250 μg/ml), and vitronectin (33 μg/ml). In other embodiments, the matrix can include 85% collagen and 15% laminin. The matrix can include other components, such as glycans, for example but not limited to heparin sulfate and chondroitin sulfate.

In certain embodiments, the composition comprises a matrix which can be a monolayer of molecules. In the process of tissue bond formation, the constituents of the matrix monolayer can acquire certain orientation and thus impart as stratifying molecular organization to the matrix. Collagen IV is particularly attractive in this application because it is non-fibrillar collagen which can act as a main framework of the matrix onto which other molecules may attach and polymerize to further form the matrix. The chemical constituents of the matrix can be formulated to optimize cellular repopulation of the wound, for example but not limited to retinal pigment epithelial cell attachment, survival and proliferation, corneal epithelial cell attachment, survival and migration in the course of healing of corneal wounds, skin epithelial cell migration and survival for closure of skin wounds, glial and/or Retinal Pigment Epithelial cell proliferation for the closure of macular and peripheral retinal holes. In certain embodiments of the inventive compositions, wherein the matrix contains photo-activated dye and collagen, after photo-activation, the composition can be used to patch different types of wounds. Non-limiting examples are ocular wound associated with or caused by: ocular disorders, trauma and surgery, ocular wounds in the anterior segment of the eye, such as cataract wounds, scleral and corneal wounds such as scleral and corneal lacerations and perforations, Bruch's membrane wounds, wounds due to penetrating keratoplasty surgery, glaucoma implants, retinal wounds and holes, retinal tears, corneal wounds after cataract surgery, deep tissue wounds, wounds to the skin, face and ocular adnexa, and orbital wounds; wounds related to the structures of the face, such as the nose and nasal sinuses and lids margins, restoring the integrity of the RPE-Bruch's membrane complex with patch grafts.

Depending upon the area of treatment, the overall thickness of the layered composition can be adjusted according to the size, including area and depth, of the wound being treated. For example, in certain embodiment wherein any one of the inventive compositions is used for the closure of corneal wounds, the overall thickness of the layered composition can be from about 10 microns to about 3000 microns, from about 10 microns to about 2000 microns, 10 microns to about 1900 microns, 10 microns to about 1800 microns, 10 microns to about 1700 microns, 10 microns to about 1600 microns, 10 microns to about 1500 microns, 10 microns to about 1400 microns, 10 microns to about 1300 microns, 10 microns to about 1200 microns, 10 microns to about 1100 microns, 10 microns to about 1000 microns. In certain other embodiments, thinner or thicker layered compositions are also contemplated. The thickness of each part of the layered composition can be from about 1 micron to about 1000 microns, 1 micron to about 900 microns, 1 micron to about 800 microns, 1 micron to about 700 microns, 1 micron to about 600 microns, 1 micron to about 500 microns, 1 micron to about 400 microns, 1 micron to about 300 microns, 1 micron to about 200 microns, 1 micron to about 100 microns, 1 micron to about 90 microns, 1 micron to about 70 microns, 1 micron to about 50 microns, 1 micron to about 30 microns, 1 micron to about 10 microns.

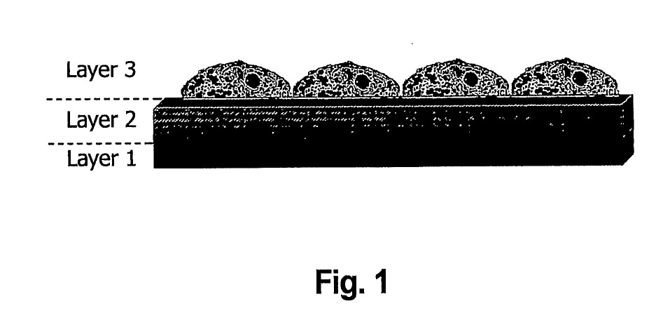

In another aspect, the invention provides a biocompatible composition, which comprises three parts: a support layer, a matrix, and a monolayer of cells. The cells can be endothelial, epithelial or mesenchymal. In certain embodiments, the cells are harvested from a donor and cultured on a culture substrate to form a monolayer. In certain embodiments, the matrix is positioned on top of the first support layer. In other embodiments, the matrix is positioned on top of an additional support layer. In other embodiments, the matrix is positioned within the additional support the layer. The additional support layer may also contain a bio-adhesive molecule. In certain embodiment, the monolayer of endothelial, epithelial or mesenchymal cells can be positioned on top of an additional support layer. The order in which the different parts of the invention are assembled to form the inventive compositions can depend on the particular application for which the composition is being used. One embodiment is demonstrated in FIG. 1. In other embodiments of the tripartite composition, which contain one support layer, the support layer can be placed between the matrix and the monolayer of cells.

The retinal pigment epithelium (RPE) is a hexagonal monolayer lining the inner aspect of Bruch's membrane (BM) that separates the neural retina from the choriocapillaris in the normal human eye. The RPE has many physiological functions, including maintenance of the blood-outer retinal barrier, phagocytosis, recycling the tips of the photo receptor outer segments, and isomerization of visual pigments. RPE cell loss occurs as a function of age. The number of RPE cells in otherwise normal human eyes decreases by approximately 0.3% per year. Dysfunction of the RPE can occur as a primary initiating event or secondary to changes in the outer retina or choriocapillaris and may play a role in a wide variety of sight-threatening diseases, including age-related macular degeneration (AMD). Because of its role in various diseases, transplantation of the RPE may be a therapeutic alternative in the management of patients with tapetoretinal degenerations, AMD, and other disorders.

Age-related macular degeneration (AMD) is the leading cause of vision loss in the United States and Western Europe. Nearly 2 million Americans over the age of 55 are diagnosed with AMD each year and the implications of visual loss in these patients are significant. AMD is expected to become even more prevalent over the coming years due to the aging of baby boomers. One estimate is that the number of people that will lose sight due to AMD will increase to 6.7 million by 2010.

There are two major types of AMD, “dry” AMD and “wet” or “exudative” AMD. “Dry” AMD is characterized by gradual loss of the RPE cells, which is followed by loss of neighboring choriocapillaris (CC) and photo receptor cells. “Dry” AMD constitutes 90% of the cases where visual loss develops gradually but at a slower pace over years. “Wet” AMD is characterized by ingrowth of choroidal vessels into the avascular subretinal space and rapid visual deterioration. Ten percent of visual loss in AMD is due to “wet” AMD, although this type of AMD can be more severe than the dry type.

Various forms of grafts and transplantation of RPE cells to the eye have been suggested. To date none of these forms constitute an effective manner for reconstructing a dystrophic retina. The transplantation of retinal cells to the eye can be traced to a report by Royo et al., Growth 23: 313-336 (1959) in which embryonic retina was transplanted to the anterior chamber of the maternal eye. A variety of cells were reported to survive, including photo receptors. (Del Cerro et al., Invest. Ophthalmol. Vis. Sci. 26: 1182-1185, 1985). Neonatal retinal tissue could be transplanted into retinal wounds. (Turner, et al. Dev. Brain Res. 26:91-104 (1986).

Prior to RPE transplantation, in cases of AMD where subretinal neovascular membranes developed, such membranes are generally removed to prevent additional subretinal edema and hemorrhaging. Several treatment modalities have been proposed for treatment of these neovascular membranes. In selected cases, therapeutic benefit has been shown with thermal laser photo-coagulation and photo-dynamic therapy (PDT) (Schmidt-Erfurth, Miller et al., 1999, Arch Ophthalmol, 117(9): 1177-87). Thermal treatment options aim to destroy the subretinal neovascular membrane, but destruction of subretinal neovascular membrane with laser treatment can also destroy the central vision. Because successful application of PDT requires multiple treatment and visual loss cannot be totally avoided, these treatments often do not restore the sight but rather slow down the loss of visual acuity. Thus, the majority of patients experience visual loss after either treatment, with concomitant scar formation and irreversible disruption of the subretinal architecture. Scar formation results in further damage to the overlying photo receptor cells (PRC) and makes it impossible to restore foveal vision. In addition, recurrent subretinal neovascular membranes are very frequent with both techniques. Laser photo-coagulation results in 52% recurrence within one year after treatment, and PDT requires multiple yearly treatments to avoid recurrence. Laser photo-coagulation and PDT are not applicable to “dry” AMD or other RPE dystrophies.

Damaged Bruch's membrane can be a critical impediment to the successful transplantation of RPE cells. During the progression of AMD, Bruch's membrane slowly loses its normal function. Bruch's membrane may also be damaged during surgical removal of choroidal neovascularization. Intrinsic Bruch's membrane damage from disease, plus removal of the inner aspects of human Bruch's membrane during subfoveal surgery, both limit the ability of transplanted RPE to attach to Bruch's membrane, proliferate and repopulate this surface. Therefore repair and/or replacement of damaged Bruch's membrane are considered an essential step of constructive subretinal surgery for AMD. The inventive compositions are suitable for use in repair and treatment of damaged Bruch's membranes, treatment of damaged tissue due to ocular disorders, trauma and surgery.

In addition, reconstitution of the extra cellular matrix components can increase RPE attachment, survival and population. For example, pre-coating inner collagen layer (ICL) of Bruch's membrane with super pharmacological doses of laminin (330 μg/ml), fibronectin (250 μg/ml), and vitronectin (33 μg/ml) has been shown to increase the attachment rate of RPE. However, survival and population of aged ICL requires cleaning of Bruch's membrane with weak non-polar detergents such as Triton-X prior to extra cellular matrix protein coating. Thus, reconstitution of transplanted RPE cells under the best conditions does not allow transplanted RPE cells to populate and function on diseased and damaged Bruch's membrane defect as effectively as on their own basal lamina. (Del Priore et al., 2000, Abstract, Assoc. for Research in Vision and Ophthalmology, Annual Conference; Geng et al., 2001, Abstract, Assoc. for Research in Vision and Ophthalmology, Annual Conference; Tezel et al., 2001, Assoc. for Research in Vision and Ophthalmology, Annual Conference).

Likewise, patching Bruch's membrane defects with either synthetic polymers or with biomembranes such as the anterior lens capsule has been ineffective. Both types of remedial efforts have been unsuccessful due to technical difficulties of implanting these membranes into the subretinal space and subsequent wrinkling and undulation due to RPE contraction (Giordano, et al, 1997, J. Biomed Mater. Res., 34(1): 87-93; Hartmann et al, 1999, Graefes Arch. Clin. Exp. Ophthalmol. 237(11): 940-5; Folke, et al. 2002, Acta Ophthalmol. Scand., 80(1): 76-81).

Rose bengal (tetratodotetrachioro fluorescein disodium salt) is a halogenated derivative of plant based fluorescein dye. Several properties of rose bengal make it suitable to be used to affix the composition of the invention on Bruch's membrane. These properties include its long term use in ophthalmology without any known toxicity, its ability to photo-activate and cross-link, the ability to easily remove excess dye in the context of a classical vitrectomy, its ability to be sterilized, and its low cost and stability. Rose bengal can be photo-activated through the generation of singlet oxygen upon light activation. This property of rose bengal has been used for several purposes, mainly the treatment of experimental preretinal neovascularization using photo-dynamic thrombosis (Wilson, Saloupis et al. 1991, Invest Ophthalmol. Vis. Sci., 32(9): 2530-5), the photo-dynamic inactivation of adenoviral vectors(Schagen, Moor et al. 1999, Gene Ther. 6(5): 873-81), the photo-dynamic cross linking of proteins (Shen, Spikes et al. 1996, J. Photochem. Photobiol. B 353 213-9) and the photo-chemical formation of keratodesmomes for repair of lamellar corneal incisions (Mulroy, Kim et al. 2000, Invest. Ophthalmol. Vis Sci. 41(11): 3335-40). Likewise, the generation of singlet oxygen upon photo-activation and resulting cross-linking of proteins has made rose bengal a desirable compound for fixing Bruch's membrane patch grafts on Bruch's membrane defects. (Tezel et al., 2002, Assoc. for Research in Vision and Ophthalmology, Annual Meeting). Other molecules, which can be used in the inventive composition, include but are not limited to glyceraldehyde, Riboflavin and Lissamine Green.

In one embodiment, the biocompatible composition comprises one part: a support layer, for example made of gelatin, wherein the support layer is also coated or impregnated with a photo-adhesive molecule, for example rose bengal, thus serving as a transport scaffold for the photo-adhesive. In another embodiment, the support layer is coated or impregnated with a chemically active molecule, for example but not limited to glyceraldehydes. In another aspect of the invention, the mono-partite composition is used to enhance cell adhesion during transplantation, wound closure and healing in various types of tissues, including but not limited to ocular wounds.

In another embodiment, the biocompatible composition comprises two parts: one part is a support layer, for example made of gelatin, wherein the support layer can also be coated or impregnated with a bio-adhesive molecule, thus serving as a transport scaffold for the photo-adhesive. Non-limiting examples of bio-adhesives are the photo-activated adhesive rose bengal and the chemically active bio-adhesive glyceraldehydes. A second part of the invention is an artificial or biological matrix, optionally processed (i.e. cleaned and coated with extra cellular matrix proteins) to enhance cell attachment, cell survival, and/or wound closure. In certain embodiments, the matrix can also be impregnated or coated with bio-adhesive molecules. In other embodiments, the matrix can be placed on an additional support layer, which can be impregnated or coated with bio-adhesive molecule. In another aspect of the invention, the bi-partite composition (i.e. the scaffold material and the matrix) is used to enhance cell adhesion during transplantation, wound closure and healing in various types of tissue, including but not limited to ocular wounds.

In another embodiment, the biocompatible composition comprises three parts: one part is a support layer, for example made of gelatin, wherein the support layer is also coated or impregnated with a bio-adhesive molecule, thus serving as a transport scaffold for the photo-adhesive. Non-limiting examples of bio-adhesives are the photo-activated adhesive rose bengal and the chemically active bio-adhesive glyceraldehydes. A second part of the invention is an artificial or biological matrix, optionally processed (i.e. cleaned and coated with extra cellular matrix proteins) to enhance cell attachment, cell survival, and/or wound closure. A third part of the invention is a monolayer of mesenchymal, epithelial, or endothelial cells, for example but not limited to retinal pigment epithelial (RPE) cells or fibroblasts. In certain embodiments, the matrix can also be impregnated or coated with bio-adhesive molecules. In another aspect of the invention, the tripartite composition (i.e. the scaffold material, the matrix, and the monolayer of cells) is used to enhance cell adhesion during transplantation, wound closure and healing in various types of tissue, including but not limited to ocular wounds. The tri-partite composition can be used specifically to repair damaged eye tissue including areas of human Bruch's membrane (BM) damaged by age-related human macular degeneration.

Any of the inventive compositions is contemplated for use in tissue bonding. In certain aspects, the compositions of the invention are used in methods for the treatment of oculars wounds associated with ocular disorders, trauma and surgery. In other aspects, the inventive compositions can be used in methods to treat wounds elsewhere within the human body, such as closure of skin wounds, vessels, and deep-tissue-layer wounds. Any one of the mono-partite, bi-partite or tri-partite compositions can be used in these methods. A non-limiting example is a composition, which comprises a support layer, such as gelatin or other collagenous mixture, impregnated with an adhesive molecule including but not limited to rose bengal or glyceraldehydes.

In another aspect of the invention, a kit is provided comprising either the mono-partite, bi-partite or tri-partite composition in a sterile package that optionally contains solutions for reconstitution. The reconstitution technique can be advantageous because by mixing the gel of the scaffold with the bio-adhesive chemical just prior to application, one can omit the unpredictable outcomes that occur due to a chemical leak or diffusion, or secondary scaffold-chemical interactions that may happen during storage. In certain embodiments, wherein the composition comprises a photo-activated adhesive molecule, the kit provides the inventive compositions in a light protected container. In other embodiments, wherein the composition comprises a chemically-active adhesive molecule, the chemically active adhesive molecule is provided separately from the rest of the components of the kit. In these embodiments, the bio-adhesive composition is formed by combining the chemically active bio-adhesive with the rest of the components in the inventive composition.

In still another aspect, the invention provides methods for enhancing wound closure using the compositions and kits in treating corneal wounds following cataract surgery, refractive surgery, penetrating keratoplasty and other applications; after glaucoma surgery, including but not limited to trabeculectomy, tube implants and others; and orbital and lid surgery, plus additional repair of eye tissue that includes trauma, ruptured globes, iris repair and other surgical repair of eye tissue.

In one aspect of the invention, the mono-partite, bi-partite or tripartite composition can be used to repair a wound in different body tissues. The compositions of the invention can be used to treat wounds in any type of body tissue, including but not limited to, skin, eye, and various internal organs.

In one aspect of the invention, the mono-partite, bi-partite tripartite composition is used specifically to repair damaged eye tissue including areas of human Bruch's membrane (BM) damaged by age-related human macular degeneration.

In certain aspects, the invention provides methods for promoting and enhancing wound closure using the compositions and kits in corneal wounds following cataract surgery, refractive surgery, penetrating keratoplasty and other applications which include surgical repair of eye tissue. In other aspects, the invention provides methods for promoting and enhancing wound closure using the compositions and kits for closure of cataract surgery wounds. In other aspects, the invention provides use of the compositions of the invention in methods for promoting and enhancing wound closure virectomy wounds. In other aspects, the invention provides methods for promoting and enhancing wound closure using the compositions and kits in wounds in the anterior segment of the eye, such as scleral and corneal lacerations and perforations. In other aspects, the invention provides methods for promoting and enhancing wound closure using the compositions and kits closure of penetrating keratoplasty wounds. In other aspects, the invention provides methods for promoting and enhancing wound closure using the compositions and kits in closure of wounds related to glaucoma implants and surgery. In other aspects, the invention provides methods for promoting and enhancing wound closure using the compositions and kits in wounds of the skin, face and ocular adnexa, and orbital wounds. In other aspects, the invention provides methods for promoting and enhancing wound closure using the compositions and kits in wounds related to the structures of the face, such as the nose and nasal sinuses and lids margins. In other aspects, the invention provides methods for promoting and enhancing wound closure using the compositions and kits to restore the integrity of the RPE-Bruch's membrane complex with patch grafts. In other aspects, the invention provides methods for promoting and enhancing wound closure using the compositions and kits in closure of wounds related to the retina including peripheral and macular retinal holes. In other aspects, the invention provides methods for promoting and enhancing wound closure using the compositions and kits in wound elsewhere within the human body such as wounds of skin, vessels, and deep tissue layers.

Accordingly, the invention provides a composition for use in promoting and enhancing wound closure comprising a transport scaffold comprising a photo-adhesive molecule. The composition may also comprise a matrix, which can be processed to enhance cell attachment and survival, and a monolayer of cells, selected from the group consisting of epithelial cells and endothelial cells. In certain embodiment of the invention, the cells are retinal epithelial cells. In certain embodiments of the invention, the bio-adhesive molecule is selected among photo-adhesive and chemically active molecules. In one embodiment, the photo-adhesive molecule is rose bengal. In another embodiment, the photo-adhesive molecule is riboflavin. In another embodiment, the photo-adhesive molecule is lissamine green. In another embodiment, the bio-adhesive molecule is glyceraldehyde.

Advantageously, the invention provides a composition wherein a bio-adhesive molecule is impregnated or coated on a transport scaffold material, which provides solid support for the bio-adhesive. Delivering the bio-adhesive agent on a solid support layer provides for accurate application of the bio-adhesive precisely to the tissues to be bonded. The solid support layer further ensures that the composition which includes the bio-adhesive is contained only within the area of the tissues to be bonded. Precise delivery the bio-adhesive is particularly important in the application of composition which comprises a chemically active bio-adhesive.

The inventive compositions, which comprise solid support layer, provide several advantages over bio-adhesive compositions know in the art. The support layer, which is coated or impregnated with bio-adhesive molecules, ensures precise application of the bio-adhesive composition to the wound area. The support layer prevents the undesirable spread of a liquid or viscous bio-adhesive composition to areas distant from location of the tissue to be bonded. Regardless of the depth of the wound, a bio-adhesive composition which comprises an artificial or biological matrix can cover the wound and create a habitable surface for the survival, proliferation and migration of transplanted and/or native cells, for example but not limited to Retinal Pigment Epithelial cell, corneal epithelium, or glial cells. Photo-activation or chemical cross-linking process can impart vertical and horizontal stability to the wounds that is necessary for proper wound healing.

In a certain embodiment, the inventive composition has two parts: a support layer which is impregnated or coated with a bio-adhesive molecule, photo-activated and/or chemically active molecule, and a matrix. The exact components of the matrix will depend on the particular application of the composition. In certain embodiments, wherein the composition is used in application such as corneal transplantation, wherein part or all of Bruch's membrane has been or will be removed from the host eye prior to the transplantation of the therapeutic composition, the matrix comprises collagen. Using the inventive compositions in application which include patching of Bruch's membrane, the layer of collagen in the matrix should be less than 100 microns in thickness, from about 1 micron to about 10 microns, from about 1 micron to about 20 microns, from about 1 micron to about 30 microns, from about 1 micron to about 40 microns, from about 1 micron to about 50 microns, from about 1 micron to about 60 microns, from about 1 micron to about 70 microns, from about 1 micron to about 80 microns, from about 1 micron to about 90 microns, from about 1 micron to about 99 microns. The collagen layer serves to anchor the RPE cells to the choroid or to the outer aspects of the Bruch's membrane, or in place of the removed Bruch's membrane as well as to inhibit subretinal neovascularization through and around the RPE. The remaining components of the matrix serve, inter alia, to support the matrix and prevent wrinkling or distortion of the matrix.

In certain embodiments, the matrix can be formed at a tissue wound, or on an intact Bruch's membrane in situ. In these instances, the matrix can comprise collagen in order to afford adequate support. Alternatively, a support layer can be placed on the tissue wound or the intact Bruch's membrane.

The matrix can be formed from dehydrated collagen by re-hydration with phosphate buffered saline to final concentration of 3.0 mg/ml. Then pH can be adjusted a physiologival pH, for example with 0.1N NaOH to pH7.4. Other extra cellular matrix proteins can be at the following concentrations: laminin (330 μg/ml), fibronectin (250 μg/ml), and vitronectin (33 μg/ml). Once all constituents of the matrix are added, they are allowed to polymerize for 1 hour at 37° C. and form the matrix. The matrix can be washed three times with phosphate-buffered saline and stored at 4° C. The matrix may also comprise other components such as pharmacologic agents including immunosuppressants such as cyclosporin A, anti-inflammation agents, such as dexamethasone, anti-angiogenic factors, anti-glial agents and anti-mitotic factors.

Photo-activation of rose bengal. Exposed outer surface of the Bruch's membrane and outer collagenous layer are be painted with rose bengal. Rose bengal solution can be prepared in phosphate buffered saline and its concentration is generally about or less than 5 mM. Rose bengal concentrations higher than 5 mM can denature collagen. In certain embodiments, the support layer is a gelatin sheet, which can be impregnated with rose bengal, or glyceraldehyde. The gelatin support layer thus supplies extra collagen between the lips of the wound and acts like a bridge when cross-linked by the photo-adhesive molecule. The gelatin sheet impregnated with rose bengal is exposed for three minutes to a white light source at 35 mW strength, which light can be optionally filtered. Optionally, a laser is used to adhere the composition to the Bruch's membrane. Because the maximum absorption of rose bengal is at 559 nm, the molar absorption kinetics can be maximized using a laser with an emission wavelength at around this wave length. This may shorten the time required to create adequate photo-adhesion, and minimize the amount of time required to create photo-adhesion. Obtaining highest photo-adhesion per quanta will decrease any possible risk for collateral damage. Appropriate wavelengths are used for other photo-activated adhesive molecules, and no exposure to light is necessary for the chemically active molecules, for example glyceraldehydes.

An inventive composition, which comprises a bio-adhesive molecule, can be used for sutureless closure of corneal and scleral incisions. In a non-limiting example, the support layer is made of gelatin sheets which can be prepared as follows: Gelatin blocks, prepared at a concentration of about 50% (weight/volume), are firm enough to manipulate during the surgery by the surgeon. Gelatin blocks with rigidity of 300 blooms (Sigma, St. Louis, Mo.) are prepared, sterilized with gamma irradiation (2.7 Megarads) and dissolved in Minimal Essential Medium (MEM, Gibco, Grand Island, N.Y.). The addition of 300 mM sucrose maintains the gelatin sheets in a solid phase at temperatures below 37° C. and permits their melting within minutes of contact with tissue at body temperature. Once the gelatin dissolves the solution is poured into 35-mm dishes (Falcon #3001, Becton Dickinson, Lincoln Park, N.J.) and is allowed to cool for 15 minutes to solidify at room temperature. Solid gelatin blocks are stored at 4° C. and used within 24 hours to minimize the time-dependent change in rigidity and melting point of the gelatin. Gelatin blocks are cut into triangular pieces and mounted on a vibratome (Series 1000, Technical Products International, St Louis, Mo.) with the basal side facing a 102-μm thick steel blade (Personna® American Safety Razor Company, Staunton, Va.). The moving platform is sterilized with ethylene oxide gas and the vibratome is cleaned with 70% alcohol. Gelatin sheets (100-μm thick) are cut from the blocks and kept in CO₂-free medium (Gibco, Grand Island, N.Y.) at 4° C. The entire procedure is performed within tissue culture hoods in a class 100 clean room. Gelatin sheets can be impregnated or coated with rose bengal solution, resterilized and packed in light-tight sterile pouches.

At the end of ocular surgery such as either a cataract extraction or vitrectomy, these packages are opened by the surgeon and a sheet impregnated with bio-adhesive, for example but not limited to rose bengal, are placed over or between the lips of the wound by the surgeon. Upon touching the ocular tissue, the gelatin melts and releases collagen and rose bengal. Short-term illumination (<3 minutes) of the wound with an endoilluminator rapidly activates rose bengal and cross-links the collagen resulting in closure of the wound. Ambient light from an operating microscope is enough to activate rose bengal.

In other embodiments, similarly cut gelatin sheets can be soaked with any one of a number of suitable bio-adhesives. Non-limiting examples are glyceraldehyde and riboflavin. Glyceraldehyde is a bio-adhesive which does not require photo-activation. Glyceraldehyde is chemically active bio-adhesive, which creates cross-links between primary amines. Crosslink formation between primary amines of adjacent tissues effectively produces a bond between these tissues. An advantage that chemically active molecules provide over photo-activated molecules is that their bio-adhesive characteristics are not affected by ambient light. Glyceraldehyde, which is a byproduct of oxidative cycle (Krebs Cycle), demonstrates low cell toxicity.

Chemically active bio-adhesive, including but not limited to glyceraldehydes, is generally packed separately from the gelatin sheet, and the sheet is coated or impregnated just prior to application to the wound site. Long-term exposure, such as presoaking the gelatin sheet with chemically active bio-adhesive, for example glyceraldehydes, can result in cross-linking of the collagen within the gelatin, which can cause a slow or delayed release of glyceraldehyde. Slow or delayed release may result in limited cross-linking of the collagen from the gelatin sheet with the exposed tissue collagen at the wound site, and decrease the efficacy of wound closure. Support layers, made of gelatin and impregnated or coated with photo-activated molecules, do not change their structure when kept inside dark packages until the time of use. For example, riboflavin-impregnated or coated gelatin sheets must be treated with 0.1% riboflavin at 4° C. in the absence of light, for 30 minutes prior to packing. Activation of riboflavin requires irradiation at 370 nm for at least 3 minutes.

In certain embodiments, the support layer comprises non-protein components that deliver the chemical adhesive. Long-term storage of such composition, wherein the non-protein support layer is impregnated or coated with glyceraldehydes, or other chemically active bio-adhesive molecules, does not affect the structure of the support layer or the activity of the chemically active bio-adhesive.

The invention also provides a method for treating a wound comprising administering an effective amount, for example in the form of an appropriately sized strip, slice or sheet, of the composition of the invention to a subject in need thereof. In one embodiment, the wound is ocular, such as a corneal wound. In another embodiment, the wound is a scleral wound. In another embodiment, the wound is an iris wound. In another embodiment, the corneal wound is after cataract surgery. In another embodiment, the corneal wound is after refractive surgery. In another embodiment, the corneal wound is after penetrating keratoplasty surgery. In another embodiment, the wound is a retinal wound, such as a retinal hole in the periphery or a macular hole. In another embodiment, the wound is a scleral and corneal laceration or perforation. In another embodiment, the wound is due to glaucoma implants and surgery. In another embodiment, the wound is of the skin, face and ocular adnexa, or orbital wound. In another embodiment, the wound is due to the structures of the face, such as the nose and nasal sinuses and lids margins. In another embodiment, the wound is due vitrectomy surgery.

The invention further provides a method for treating ocular disorders comprising administering an effective amount of the composition of the invention to a subject in need thereof. In certain embodiments of the invention, the wound is due to an ocular disorder. In another embodiment of the invention, the ocular disorder can cause tissue damage that requires vascular and neural grafting, and stabilization of therapeutic prosthetic or implantable devices. In another embodiment, the wound is a defect or damage in Bruch's membrane. In one embodiment, the ocular disorder is age-related macular degeneration. In another embodiment, the ocular disorder is a disorder affecting the RPE-Bruch's membrane complex. In another embodiment, the ocular disorder is presumed ocular histoplasmosis syndrome. In still another embodiment, the ocular disorder is myopic maculopathy. In still another embodiment, the disorder is ingrowth of neovascularization form another disorder affecting Bruch's membrane.

The following non-limiting examples illustrate the invention, and are set forth to aid in the understanding of the invention, and should not be construed to limit in any way the scope of the invention as defined in the claims which follow thereafter.

### Examples

### Example 1

### Preparation of Bruch's Membrane

Bruch's membrane explants were harvested from cadaver eyes. RPE-Bruch's membrane-Choroid complexes were dissected out under a microscope; washed with PBS three times and kept in 0.02N ammonium hydroxide for 30 minutes to lyse all cellular components. Three cycles of PBS washing followed by removal of the basal lamina mechanically as described before. Briefly, a full thickness circumferential incision was made posterior to the ora serrata and the anterior segment and vitreous were removed carefully. The posterior pole of each eye cup was inspected visually with direct and retroillumination under a dissecting microscope and globes were discarded if there was any evidence of subretinal blood, previous surgery or any extensive structural or vascular alteration of the posterior segment due to a disease process, such as proliferative diabetic retinopathy or proliferative vitreoretinopathy. The eyecups were put in carbon dioxide-free Media (Gibco) and a scleral incision was made 3 mm from the limbus and extended circumferentially. Four radial incisions were then made and the sclera was peeled away. A circumferential incision was made into the subretinal space 1 mm posterior to the ora serrata. The choroid-Bruch's membrane-RPE complex was then carefully peeled towards the optic disc and removed after trimming its attachment to the optic nerve. Native RPE were removed by bathing the explant with 0.02 N ammonium hydroxide in a 50-mm polystyrene Petri dish (Falcon, Becton Dickinson, Lincoln Park, N.J.) for 20 minutes at room temperature followed by washing three times in phosphate-buffered saline (PBS).

The Bruch's membrane explant from the fellow eye was prepared by removing the RPE with 0.02N ammonium hydroxide as described above. The Bruch's membrane explant was then floated in CFM over a 12-18 micron thick hydrophilic polycarbonate-polyvinylpyrrolidone membrane with 0.4 micron pores (Millipore, Bedford, Mass.) with the basal lamina facing towards the membrane. The curled edges were flattened from the choroidal side with fine forceps without touching Bruch's membrane. Four percent agarose (Sigma Chemical Co., St. Louis, Mo.) was poured on the Bruch's membrane-choroid complex from the choroidal side and the tissue was kept at 4° C. for 2-3 minutes to solidify the agarose. The hydrophilic membrane was peeled off along with the basal lamina of the RPE thus exposing the bare inner collagenous layer. 6 mm circular buttons were then trephined from peripheral Bruch's membrane on a Teflon sheet and placed on 4% agarose at 37° C. in non-treated polystyrene wells of a 96 well plate (Corning Costar Corp., Cambridge, Mass.). The agarose solidified within 2-3 minutes at room temperature, thus stabilizing the Bruch's membrane explant. The wells were gently rinsed with PBS three times for 5 minutes, gamma sterilized (20,000 rads) and then stored at 4° C.

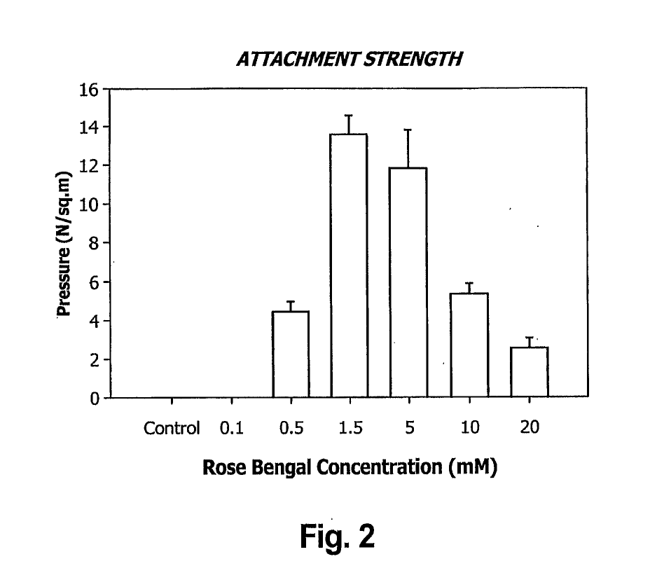

Explants with exposed ICL were then cut with a 6-mm trephine and stored in sterile PBS after gamma sterilization at 20,000 rads. At the time of use ICL surface was painted with rose bengal (at concentrations as provided in FIG. 2) and two patches were faced to each other at their ICL surface. Extra fluid was removed with a Whatman #30 filter paper and the two surfaces were apposed. Bruch's membrane patches were exposed to 35 mW cold halogen light at an incandescence level comparable to vitrectomy endo illuminator (68 candela/sq. mt). Exposure time was limited to three minutes not to exceed photo-toxicity thresholds.

### Example 2

### Retinal Pigment Epithelial (RPE) Cells

Primary RPE cultures were prepared from the posterior poles of human cadaver eyes. The eyes were cleaned of extraocular tissue. The space between the sclera and choroid (i.e. the suprachroidal space) of the posterior pole was sealed by sticking the choroid and sclera together with cyanoacrylate glue and a small scleral incision was made 3 mm posterior to the limbus until the choroidal vessels are exposed. Tenotomy scissors were introduced through the incision into the suprachoroidal space and the incision was extended circumferentially. Four radial relaxing incisions were made in the sclera and the sclera is peeled away from the periphery to the optic nerve with care to avoid tearing the choroid. The eye cup is then incubated with 25 u/ml of Dispase (Gibco, Grand Island, N.Y.) for 30 minutes, rinsed with carbon dioxide-free medium (CFM, Gibco) and a circumferential incision was made into the subretinal space along the ora serrata. The loosened RPE sheets-were collected with a Pasteur pipette and plated onto bovine corneal endothelium-extracellular matrix (BCE-extracellular matrix) coated 60 mm treated plastic dishes (Falcon, Becton-Dickinson, UK, Plymouth, England). The cells were incubated in a humidified atmosphere of 5% CO₂ and 95% air at 37 C and maintained in Dulbecco's modified Eagle's medium (DMEM H16, Gibco) supplemented with 15% fetal bovine serum (FBS), 100 IU/ml penicillin G, 100 mg/ml streptomycin, 5 mg/ml gentamicin, 2.5 mg/ml Amphotericin B and 1 ng/ml human recombinant basic fibroblast growth factor ((bFGF) to promote RPE cell growth. The medium was changed every other day and the cells observed daily. Confluent cultures are passaged by trypsinization. Cells were stained using a pancytokeratin antibody to verify that all cells are of epithelial origin.

### Example 3

### Tissue Bonding of Explants from Inner Collagenous Layer

Six-millimeter explants of human peripheral inner collagenous layer were prepared from 10 aged (>60 years-old) cadaver eyes. Exposed inner collagenous layers were painted with rose bengal and photo-melded to each other by photo-exciting rose bengal with a white light source. The light intensity matched the vitrectomy endo illuminator and exposure was limited to 3 minutes to avoid retinal photo-toxicity. Attachment strength was measured in a buoyancy chamber using different concentrations of rose bengal (0.1-20 mM). Attached grafts were kept under 0.21 mN (milliNewtons) of constant traction for 3 days to test the stability of the melding process.

### Example 4

### Application of Composition to Bruch's Membrane

The resulting matrix is placed on the surface of harvested Bruch's membrane. The matrix is attached to Bruch's membrane using concentrations of rose bengal varying between 0.1-20 mM. Attachment strength was measured in Bruch's membrane patches.

### Example 5

### Determination of Optimum Concentration of Rose Bengal

Three minutes of exposure to 35 mW of light at a distance of 40 millimeters resulted in photo-melding of exposed human inner collagenous layers at concentrations above 0.1 mM. Attachment strength increased up to 14.1 N/m² at 1.5 and 5 mM concentrations of rose bengal. Surprisingly, at higher concentrations attachment strength decreased (5.7 N/m² and 2.83 N/m² at 10 and 20 mM, respectively) due to collagen denaturation (FIG. 2). The minimum concentration to attain strong photo-melding of human inner collagenous layer grafts to each other was 1.5 mM. Photo-melded grafts remained attached during the 72 hours of observation at 37° C. in spite of continuous traction.

### Example 6

### Photo-Activation

Three minutes of exposure to 35 mW of light at a distance of 40 millimeters resulted in photo-melding of exposed human inner collagenous layers at concentrations above 0.1 mM. Photo-melded grafts remained attached during the 72 hours of observation at 37° C. in spite of continuous traction. (See FIG. 2)

### Example 7

### Attachment of RPE Cells

First passage RPE cells from a human donor were harvested as soon as the cells reached confluence to minimize the effects of culture age on the colorimetric assay described below. This assay indirectly estimated the number of live cells by measuring intracellular dehydrogenase activity (CellTiter 96™ Aqueous non-radioactive cell proliferation assay, Promega, Madison, Wis.). Dehydrogenase enzymes found in live cells reduce MTS (3-(4,5-dimethylthiazol-2-yl)-5-(3-carboxymethoxyphenyl)-2-(4-sulfophenyl)-2H-tetrazolium) into the aqueous soluble formazan in the presence of an electron coupling agent (phenazine methosulphate, PMS). The quantity of formazan product was determined from the absorbance at 490 nm and is directly proportional to the number of living cells in culture.

Confluent RPE cells cultures were synchronized by placing them in serum and phenol-free MEM (Modified Eagle's Medium; Gibco, Grand Island, N.Y.) for 24 hours prior to treating with 0.24% trypsin/0.25% EDTA in HBSS for 10 minutes. Two milliliters of 0.1 mg/ml aprotinin (Sigma, Saint Louis) in HEPES (pH=7.5) (N-(2-hydroxyethyl)piperazine-N′-(2-ethanesulfonic acid); 4-(2-hydroxyethyl)-1-piperazineethanesulfonic acid) is used to quench the trypsin reaction and the cell suspension is centrifuged for five minutes at 800 rpm. The cell pellet was then washed three times, triturated to yield a single cell suspension and then resuspended in phenol red-free MEM without serum. Cell number was determined by using a Coulter Counter (Model Z-1, Coulter Scientific, Hialeah, Fla.) and cell viability is assessed using the Live/Dead Viability Kit (Molecular probes, Portland, Oreg.). At least 250 cells were examined under 100×magnification and the viability was expressed as the average ratio of live cells to the total number of cells in three randomly chosen areas. Fifteen thousand viable RPE cells were plated on different layers of Bruch's membrane explants and serum-free MEM containing 100 IU/ml penicillin G, 100 mg/ml streptomycin, 5 mg/ml gentamicin and 2.5 mg/ml Amphotericin B were added to reach a final volume of 200 ul in each well. At this plating density, the RPE cells should cover approximately 15% of the plating area assuming a cell diameter of 20 um. Positive and negative controls are performed each time the attachment assay is run with RPE cells plated onto tissue culture plastic serving as the positive control of RPE cells plated on 4% agarose serving as the negative control. Cells are allowed to attach to the surface in serum-free MEM for 24 hours in a humidified atmosphere of 95% air/5% Co2 at 37 C. Unattached cells were removed from the tissue culture plates by gently washing the wells three times with MEM.

### Example 8

### Assay of RPE Cells Number

MTT assay. The number of RPE cells reattached to Bruch's membrane in organ culture was assayed with the MTT assay. MTT [3-(4,5-Dimethylthiazol-2-yl)-2,5-diphenyltetrazolium Bromide] (Sigma, St. Louis) is a dye whose absorption characteristics change when it is dehydrogenated by cellular mitochondrial dehydrogenase, the activity of this latter enzyme is proportional to the number of live cells exposed to the dye. Thus, the use of MTT allows determination of the number of live cells attached to Bruch's membrane. The amount of yellow reduced tetrazolium is quantified with an ELISA reader with a 570 nm filter. The solid tissue is removed from the wells containing explants and the 96-well plates are then read with an ELISA reader. The number of cells attached to the surface is then calculated by comparing the ELISA readings obtained on the wells with an unknown number of cells to a standardized curve. Statistical analysis: Triplicate wells are used to calculate the average reattachment rate to each layer of Bruch's membrane. Data from all experiments are pooled and expressed as mean±standard deviation. The reattachment, apoptosis, proliferation rates and mitotic indices on different substrates between young and old donors are compared by Mann-Whitney-U test and the differences between the mean rates of various, groups are analyzed in pairs by Dunn's multiple comparison test. A confidence level of p<0.05 is considered to be statistically significant.

Apontosis Rate. Twenty-four hours after plating onto different layers of B Bruch's membrane, wells were washed gently three times with MEM and fixed with 4% paraformaldehyde for four hours. Apoptotic cells are identified using a TUNEL stain. For this purpose, cells were permeabilized by treating with 0.2% Triton-X in 0.2 M sodium citrate solution at 4° C. for four minutes. Explants were washed three times with PBS and incubated with a solution of DNA polymerase and dUTP tagged with fluorescein for one hour. Wells were then washed three times and the percentage of cells with DNA breaks was determined under a fluorescence microscope. The total number of attached RPE cells on each layer of Bruch's membrane was estimated by trypsinizing and counting the attached RPE cells on a different set of explants. The apoptosis rate on each layer of Bruch's membrane was defined as the ratio of apoptotic cells to the total number of attached cells on that layer.

Proliferation Rate. Twenty four hours after plating, cell proliferation was stimulated by replacing the medium with MEM supplemented with 15% FBS and 1 ng/ml of recombinant human basic fibroblast growth factor (bFGF, Gibco). The number of cells on each explant is determined using the MTT assay 24 hours after growth stimulation. The proliferation rate is defined as the ratio of the number after growth stimulation to the initial number of viable cells on explant.

Mitotic Index. Twenty-four hours after inducing proliferation with FBS and bFGF, explants are fixed with 4% paraformaldehyde for four hours at room temperature and stained for nuclear Ki-67 antigen that is expressed by the proliferating cells. Wells are washed three times with PBS and incubated with 3% BSA in PBS for one hour at 37° C. to block non-specific binding sites, washed with PBS for five minutes, and incubated with 1:100 dilution of an antibody against Ki-67 (Novocastra Labs, UK). Visualization is achieved with a secondary antibody against mouse IgG tagged with Cy3 (Sigma). Cells expressing Ki-67 antigen are counted under a fluorescence microscope and the mitotic index (ratio of Ki-67 expressing cells to the total number of viable cells 24 hours after growth stimulation) is determined by examining all the cells in each well

Ability to Repopulate. Cells were fed 200 ul of MEM containing inert fluorescent beads (Lumafluor, Stony Point, N.Y.) after they reattach to the surface and supplemented with 15% FBS, 100 IU/ml penicillin G, 100 mg/ml streptomycin, 5 mg/ml gentamicin, 2.5 mg/ml Amphotericin B and 1 ng/ml of recombinant human bFGF. The culture medium was changed every other day and cell growth was monitored daily for up to 21 days using an upright fluorescence microscope (EH-2, Olympus, Japan). Fluorescence microscopy is used to determine when surface is repopulated with a confluent monolayer of RPE cells. Explants are examined by light microscopy, SEM and TEM, by protocols known in the art.

### Example 9

### Photo-Melding Process

Cellular viability of photo receptor cells (PRC), choriocapillaris (CC) and RPE cells were determined with the Live-Dead assay (Molecular Probes, Portland, Oreg.) to determine the safety of the photo-melding procedure. Human cadaver eyes were obtained from an eye bank. Anterior segments were removed and sensory retina and RPE-CC complex were dissected out. Tissue samples of 6-mm size were cut from each layer and placed in 1.5 mM and 10 mM rose bengal solution. Using similar parameters photo-melding conditions were mimicked. Viability was assessed immediately after exposure. Samples exposed to similar lighting conditions in PBS were taken as control.

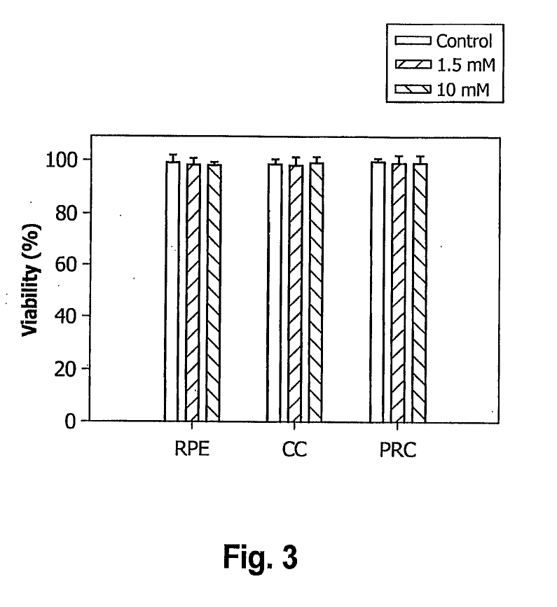

Results of viability assessment immediately after photo-melding process are shown in FIG. 3. FIG. 3 demonstrates that there is no adverse side effect on PRC, CC and RPE cell viability immediately after the procedure at both 1.5 mM and 10 mM concentrations of rose bengal.

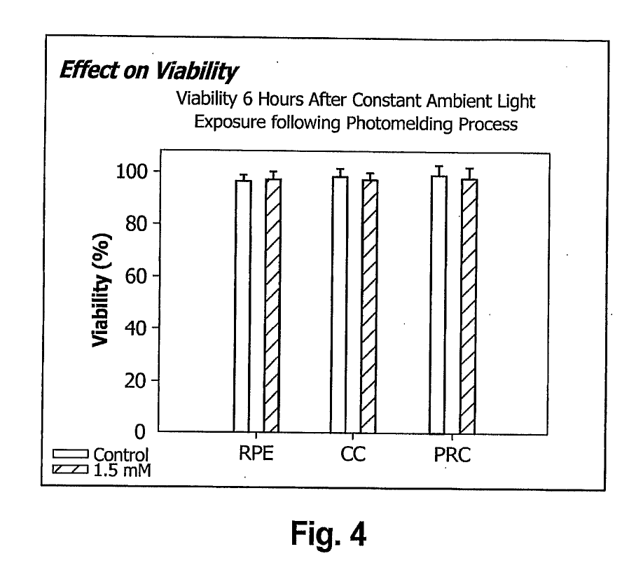

In order to assess the additive effect of subsequent ambient light on cell toxicity anterior segments of three eyecups were removed and a 20-D PMMA PC IOL was sutured into the ciliary sulcus. Sensory retina was carefully lifted off and foveal RPE cells were removed mechanically. Exposed Bruch's membrane was intentionally damaged and a 6-mm Bruch's membrane patch graft photo-melded by photo-exciting 1.5 mM of rose bengal through sensory retina. After the photo-melding procedure, the eyecup was maintained at room temperature for 6 hours exposed to ambient light. At the end of this period cellular viability was assessed with Live-Dead assay. Again, PBS treated eyes served as control. FIG. 4 demonstrates the effects of long-term ambient light exposure after photo-melding process on cell PRC, CC and RPE viability. Overall, ambient light exposure after photo-melding with 1.5 mM of rose bengal did not effect the viability of PRC, CC and RPE cells.

### Example 10

### Rose Bengal Photo-Melding Procedure Induces Ultrastructural Alterations of Bruch's Membrane

Bruch's membrane explants were photo-melded with varying concentrations of rose bengal, ranging from 0.1 to 20 mM. Transmission electron microscopy (TEM) and scanning electron microscopy (SEM). This analysis demonstrated that with the increase of the concentration of rose bengal (starting at 0.1 mM and increasing to 20 mM), there was an increasing trend of collagen crosslinking that reached its maximum at 1.5 mM. At this concentration, cross linked collagen fibers twisted onto themselves and formed macro bundles. At concentrations higher that 1.5 mM, attenuation of individual collagen fibers and frequent breaks lead to the disruption of collagen framework resulting in hollow spaces at 10 and 20 mM concentrations. Starting from 0.1 mM concentration, the number of electron-dense collagen fibers increased as the concentration of rose bengal increased, possibly due to increased oxidation and subsequent better attraction of positively charged lead and osmium on the collagen fiber. At concentrations higher than 10 mM electron dense haze started to accumulate around the collagen debris.

### Example 11

### Wound Closure and Healing

Gelatin blocks were prepared by the following method: Gelatin (300 Blooms, Sigma, St. Louis, Mo.) was dissolved in Minimal Essential Medium (MEM, Gibco, Grand Island, N.Y.), 300 mM sucrose was added and the solution was sterilized with gamma irradiation (2.7 Megarads). Addition of 300 mM sucrose maintained the gelatin sheets in a solid phase at temperatures below 37° C. and allowed the gelatin sheet to melt within minutes at body temperature once the gelatin sheet were placed over the wound. Gelatin blocks were prepared at a concentration of about 50% (weight/volume of MEM), which were firm enough to manipulate during the surgery by the surgeon. These gelatin blocks have rigidity of 300 blooms. Once the gelatin/sucrose solution in MEM was sterilized, the solution was poured into 35-mm tissue culture dish (Falcon #3001, Becton Dickinson, Lincoln Park, N.J.) and allowed to cool for 15 minutes to solidify at room temperature. Solid gelatin blocks were stored at 4° C. and used within 24 hours to minimize the time-dependent change in rigidity and melting point of the gelatin. Gelatin sheets prepared by this method can be stored at 4° C. for at least 6 months.

Gelatin blocks were cut into triangular pieces and mounted on a vibratome (Series 1000, Technical Products International, St Louis, Mo.) with the basal side facing a 102-μm thick steel blade (Personna® American Safety Razor Company, Staunton, Va.). The moving platform was sterilized with ethylene oxide gas and the vibratome will be cleansed with 70% alcohol. Gelatin sheets of about 100-μm thickness were cut from the blocks and kept in CO₂-free medium (Gibco, Grand Island, N.Y.) at 4° C. The entire procedure was performed within tissue culture hoods in a class 100 clean room.

Separate gelatin sheets were then impregnated with rose bengal (1.5 mM), glyceraldehyde (0.2 M) or riboflavin (0.1%) solution. Triplicate sets of these sheets were tested in their ability to close 3 mm corneal wounds created in freshly enucleated (<6 hrs) porcine eyes. After cleaning the globes from extraocular tissues they were inspected for the presence of any wounds. Intact globes were then mounted on a suction cup and stabilized by applying suction to the posterior pole of the globes. A 23G needle attached to a closed infusion bottle was used to enter the globe through the cornea. After ensuring that there were no leaks in the system an ocular wound was created with a surgical knife used to create cataract incisions and the absence of wound leaks was demonstrated by painting the wound site with fluorescein under blue light (Seidel test).

Gelatin sheets containing rose bengal (1.5 mM), glyceraldehyde (0.2 M) or riboflavin (0.1%) solutions were placed over the wound. Rose bengal was activated with an endoilluminator light pipe for 3 minutes. A similar illumination was applied on riboflavin-soaked gel with an UV lamp (370 nm). No illumination was applied on glyceraldehydes-soaked gels. After three minutes the intraocular pressure was increased gradually by elevating the bottle height. After each elevation, the treated wound was checked for the presence of any possible wound leak with the Seidel test, and the highest intraocular pressure at which the wound remained intact was recorded. This experiment was repeated three times on three different occasions and the results were calculated as average±standard deviation. Rose bengal withstood the highest intraocular pressures (75.6±5.8 mmHg), followed by glyceraldehyde (66.1±6.0 mmHg) and riboflavin (41.1±7.0 mmHg). PBS-soaked collagen sheets were taken as control. Both light-illuminated (10.6±3.9 mmHg) and non-illuminated (9.4±3.9 mmHg) PBS-soaked collagen shields failed to close the ocular wounds.

All publications referenced herein are hereby incorporated in their entirety. While the foregoing invention has been described in some detail for purposes of clarity and understanding, it will be appreciated by one skilled in the art, from a reading of the disclosure that various changes in form and detail can be made without departing from the true scope of the invention in the appended claims.

1. A composition comprising:
at least one support layer impregnated or coated with a bio-adhesive agent.

2. The composition of claim 1, wherein the support layer is coated on one side.

3. The composition of claim 1, wherein the support layer is coated on both sides.

4. A composition comprising:
(a) at least one support layer impregnated or coated with a bio-adhesive agent, and further comprising
(b) an additional support layer, which comprises a matrix which facilitates wound healing.

5. A composition comprising:
a) at least one support layer impregnated or coated with a bio-adhesive agent, and further comprising
b) a matrix which facilitates wound healing.

6. The composition of any one of claims 1, 4, or 5, wherein the thickness of the support layer is from about 1 micron to about 1000 microns.

7. The composition of any one of claims 1, 4, or 5, wherein the support layer comprises material selected from the group consisting of: gelatin, collagen, poly(ethylene glycol)-block-poly(epsilon-caprolactone)-block-poly(DL-lactide), PEG-PCL-P(DL) lactic acid, RGD-containing peptides (Arg-Gly-Asp) on a polyvinyl alcohol (PVA) surface or glycol-polymer matrix, heparin, alginate cross linked gels, agarose hydro-gels, and any combination thereof.

8. The composition of any one of claim 1, 4, or 5, wherein a support layer comprising gelatin further comprises sucrose.

9. The composition of any one of claims 4 or 5, wherein the matrix comprises molecules selected from the group consisting of: laminin, collagen, fibronectin, vitronectin, and any combination thereof.

10. The composition of any one of claims 4 or 5, wherein the matrix comprises amniotic membrane, human sclera, human cornea, or other basement membranes.

11. The composition of any one of claims 4 or 5, wherein the matrix consists of 85% collagen and 15% laminin.

12. The composition of any one of claims 4 or 5, wherein the matrix has a thickness from about 1 micron to about 500 microns.

13. The composition of any one of claims 4 or 5, wherein the matrix has a thickness from about 1 micron to about 1000 microns.

14. The composition of any one of claims 1, 4, or 5, wherein the composition further comprises a monolayer of epithelial cells.

15. The composition of claim 14, wherein the epithelial cells are retinal pigment epithelial (RPE) cells.

16. The composition of any one of claims 1, 4, or 5, wherein the composition further comprises a monolayer of endothelial cells or mesenchymal cells.

17. (canceled)

18. The composition of any one of claims 1, 4, or 5, wherein the bio-adhesive agent is selected from the group consisting of photo-activated molecules or chemically-active molecules.

19. The composition of any one of claims 1, 4, or 5, wherein the bio-adhesive agent is a photo-activated molecule, which is selected from the group consisting of: flavins, xanthenes, thiazines, porphyrins, chlorophyllin and photo-activated derivatives thereof.

20. The composition of claim 19, wherein the flavin photo-adhesive agent is selected from the group consisting of: riboflavin, riboflavin-5-phosphate, flavin mononucleotide, flavin adenine dinucleotide, flavin guanine nucleotide, flavin cytosine nucleotide, and flavin thymine nucleotide.

21. The composition of claim 19, wherein the xanthene photo-adhesive agent is rose Bengal or crythrosine.

22. The composition of claim 19, wherein the thiazine photo-adhesive agent is methylene blue.

23. The composition of claim 19, wherein the porphyrin photo-adhesive agent is selected from the group consisting of: protoporphyrin I through protoporphyrin IX, coproporphyrins, uroporphyrins, mesoporphyrins, hematoporphyrins and sapphyrins.

24. The composition of any one of claims 1, 4, or 5, wherein the agent is chemically-active adhesive molecule, which is selected from the group consisting of: D-glyceraldehyde, L-glyceraldehyde, glyceraldehydes-3-phosphate, glutaraldehyde, glycoaldehyde, oxoaldehydes such as glyoxal and methylglyoxal, dihydroxyacetone, threose, D-xylose, D-ribose, D-fructose, D-glucose, poly(acrylates), chitosan, cellulose derivatives, hyaluronic acid derivatives, pectin and traganth, starch, poly(ethylene glycol), sulfated polysaccharides, carrageenan, Na-alginate, gelatin, and theorems.

25. A method for promoting tissue bond formation between separate tissues, the method comprising:
a) providing a composition which comprises a support layer impregnated or coated with a tissue bonding agent,
b) applying the composition to tissues to be bonded, and
c) optionally applying electromagnetic energy to the composition to promote tissue bond formation.

26. The method of claim 25, wherein the tissues to be bonded are in the eye.

27. The method of claim 26, wherein the tissues to be bonded are in an ocular wound due to trauma, surgery, transplantation, disorder or disease.

28. The method of claim 27, wherein the ocular wound is corneal wound, iris wound, scleral wound, an anterior wound following glaucoma surgery, ocular adnexa wound, orbital wound, trabeculectomy, wound produced by tube implants, virectomy incision wound, subretinal fluid drainage wound, orbital surgery wound, lid surgery wound, scleral laceration or perforation, corneal laceration or perforation, wounds due to glaucoma implants and surgery, wounds due to the structure of the sinuses and lid margins, wound due to damage or defects in the integrity of the retinal pigment epithelial-Bruch's membrane complex.

29. The method of claim 28, wherein the corneal wound is cataract surgery wound, penetrating or lameral keratoplasty surgery wound, scalpel or laser-induced refractive surgery wound.

30. The method of claim 28, wherein the retinal wound is retinal hole in the periphery, retinal hole in the macula, or a combination thereof.

31. The method of claim 25, wherein the tissues to be bonded are in skin, in blood vessels, or in deep tissue layers.

32. (canceled)

33. (canceled)

34. The method of claim 25, wherein the disorder is selected from the group consisting of: age-related macular degeneration, disorder affecting the RPE-Bruch's membrane complex, presumed ocular histoplamosis syndrome, myopic maculopathy, and ingrowth of revascularization from a disorder affecting Bruch's membrane.

35. A method for transplantation of retinal pigment epithelial cells to a Bruch's membrane of a host's eye, the method comprising:
a) obtaining retinal pigment epithelial cells from a donor tissue;
b) applying the composition of any one of claims 1, 4, or 5 to host's Bruch's membrane,
c) positioning the retinal pigment epithelial cells of step (a) onto the composition of step (b), and
d) bonding the composition of step (b) to host's Bruch's membrane.

36. The method of claim 35, wherein the retinal pigment epithelial cells are harvested from the donor are cultured on a culture substrate to form a monolayer.

37. A kit comprising the composition of any one of claims 1, 4, or 5 dispensed into light-impenetrable container, and a pharmaceutically acceptable carrier.

38. A kit comprising the composition of any one of claims 1, 4, or 5, wherein the chemically active bio-adhesive molecule is provided separately from the remaining components of the composition.

39. A kit comprising the composition of any one of claims 1, 4, or 5 and a pharmaceutically acceptable carrier, wherein the area of the support layer is predetermined.

40. A method for making a bio-adhesive composition, the method comprising:
a) providing a gelatin block comprising about 50% gelatin;
b) sectioning a gelatin sheet from the gelatin block; and
c) impregnating the gelatin sheet with a bio-adhesive agent, thereby creating a bio-adhesive composition.

41. The method of claim 40, wherein gelatin has rigidity of 175 Blooms, 225 Blooms or 300 Blooms.

42. The method of claim 40, wherein the bio-adhesive agents is selected from the group consisting of photo-activated or chemically-active molecules.
